(19) United States
(12) Patent Application Publication
(10) Pub. No.: US 2016/0361206 A1
(43) Pub. Date: Dec. 15, 2016

(54) WASHABLE ABSORBENT COMPOSITE MATERIAL

(71) Applicant: Erin Engelhardt, Ottawa (CA)

(72) Inventor: Erin Engelhardt, Ottawa (CA)

(21) Appl. No.: 15/177,720

(22) Filed: Jun. 9, 2016

Related U.S. Application Data
(60) Provisional application No. 62/175,101, filed on Jun. 12, 2015.

(51) Int. Cl. A61F 13/15; A61F 13/14; A61F 13/47; B32B 5/02; A61F 13/49; A61F 13/539; A61F 13/62; A61F 13/00; A61F 13/472

(52) U.S. Cl. CPC A61F 13/15203; A61F 13/00068; A61F 13/141; A61F 13/15642; A61F 13/47; A61F 13/472; A61F 13/49; A61F 13/539; A61F 13/62; B32B 5/02; B32B 5/028; A61F 2013/15016; A61F 2013/15276; A61F 2013/15487; A61F 2013/4587

(57) ABSTRACT

A washable absorbent composite material having a first layer of fluid permeable fabric and a second layer of fluid permeable fabric, the layers of fabric attached by longitudinal channel binding to form channels therebetween. An absorbent core material can be received in the channels to add absorbency to the material. The absorbent composite material can be used in the manufacture of absorbent personal hygiene products.

### CROSS-REFERENCE TO RELATED APPLICATION

This application is a Utility Patent application based on a previously filed U.S. Provisional Patent application, U.S. Ser. No. 62/175,101 filed on Jun. 12, 2015, the benefit of the filing date is hereby claimed under 35 U.S.C. §119(e) and incorporated herein by reference.

### FIELD OF THE INVENTION

The present invention pertains to a washable absorbent composite material. More particularly, the present invention pertains to an absorbent composite material useful for absorbing body fluids.

### BACKGROUND

Absorbent hygiene pads act as moisture barriers to absorb moisture or body fluid. Absorbent pads also serve to enhance the comfort of a user or patient by absorbing such moisture or fluid so that the user is kept relatively dry. Absorbent articles, such as diapers, training pants, and adult incontinence undergarments, absorb and contain body exudates and prevent body exudates from soiling, wetting, or otherwise contaminating clothing or other articles that come in contact with the wearer. A wide variety of absorbent articles designed for the absorption of body fluids are known. Some examples of absorbent articles include diapers for infants and young children, incontinence garments ranging in size and shape from small hygiene pads to full diapers for children and adults, bedding pads, furniture pads, breast pads and menstrual pads.

Absorbent articles are typically comprised of a number of layers of material. These generally include, from top to bottom, a fluid permeable layer near the skin of a user to enable fluid to pass through, an absorbent layer to absorb fluid, and a fluid repellent layer to prevent leakage of fluid outside of the absorbent article. Additional layers may also be interposed between any of these layers to provide additional absorbance. The layers are generally held together by stitching, adhesives, crimping, fusing, and other methods known in the art, depending on the materials used for each layer.

Reusable absorbent pads need not be disposed of after each use, but can be washed or laundered and used again. This reduces waste and provides the user with higher quality fabrics to put next to skin. Disposable materials have also been reported to cause allergic reactions resulting in itchiness, odour and/or discharge. Construction of washable absorbent materials should take into consideration comfort, durability, absorbance and washability of the materials such that they can be repeatedly reused.

U.S. Pat. No. 5,085,653 to Levy describes a reusable incontinence underpad having a first layer of woven or knit fabric, a second layer of non-woven felt, a third layer of polyurethane film, and a fourth layer of woven or knit fabric. The first and second layers are quilted together to form a fluid absorbing portion, and the third and fourth layers are laminated together to form a fluid barrier.

U.S. Pat. No. 6,221,460 to Weber et al. describes an absorbent material for a disposable undergarment having a plurality of peaks across its surface at spaced-apart intervals forming air channels therebetween. The peaks are formed by either by joining a facing layer to itself along folds comprising absorbent material to form spaced-apart peaks, or by attaching a facing layer overtop an absorbent material having individually formed peaks at spaced-apart intervals. The peaks serve to provide air channels to separate feces from the skin in a disposable article.

U.S. Pat. No. 8,629,316 to Waxman et al. describes a reusable absorbent article having a hydrophilic top layer, a soaking layer and a liquid impermeable layer secured together to form a unitary structure. The soaking layer is a non-woven fabric having a plurality of hydrophobic polyester resin fibres having an non-circular cross section.

There remains a need for a reusable washable absorbent material that can absorb and disperse moisture to maximize the comfort of the user.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a washable absorbent composite material that can act as a moisture barrier to absorb moisture or body fluid.

In accordance with an aspect, there is provided a washable absorbent composite material comprising a first layer of fluid permeable fabric, a second layer of fluid permeable fabric, longitudinal channel binding attaching the first layer to the second layer forming channels therebetween, and an absorbent core in at least one of the channels.

In accordance with an embodiment, the fluid permeable fabric of the first layer and the fluid permeable fabric of the second layer are absorbent.

In accordance with another embodiment, the absorbent core comprises at least one filament of absorbent fibre, synthetic sponge, felt, desiccant, absorbent particulate, or a combination thereof.

In accordance with another embodiment, the washable absorbent composite material further comprises at least one additional layer of absorbent fabric between the first layer and the second layer.

In accordance with another aspect, there is provided a washable absorbent article comprising an absorbent composite material comprising a first layer of fluid permeable fabric and a second layer of absorbent fabric, the first layer and second layer attached with longitudinal channel binding forming channels therebetween, and a fluid repellent backing layer bonded thereto.

In accordance with another embodiment, at least one of the channels comprises an absorbent core.

In accordance with another embodiment, the absorbent core comprises synthetic sponge, felt, one or more filaments of absorbent fibre, desiccant, or a combination thereof. In accordance with another embodiment, the article further comprises a mesh layer on top of the first layer, wherein the channel binding extends through the mesh layer

In accordance with another embodiment, the washable absorbent article further comprises at least one additional layer of absorbent fabric between the first layer and the backing layer.

In accordance with another embodiment, the backing layer is substantially impermeable to fluid.

In accordance with another embodiment, the article is a menstrual pad, incontinence pad, feminine hygiene pad, infant diaper, child diaper, training pant, adult diaper, wound dressing, bandage, lactation pad, or incontinence undergarment.

In accordance with another aspect, there is provided a washable absorbent composite material comprising a first layer of fluid permeable fabric; a second layer of fluid permeable fabric; longitudinal channel binding attaching the first layer to the second layer forming channels therebetween; and a fluid repellent backing layer bonded to the second layer.

In accordance with an embodiment, the composite material comprises a mesh layer on top of the first layer, wherein the channel binding extends through the mesh layer.

In accordance with another embodiment, the composite material comprises an absorbent core in at least one of the channels.

In accordance with an embodiment, the absorbent core comprises at least one filament of absorbent fibre, synthetic sponge, felt, desiccant, absorbent particulate, or a combination thereof.

### BRIEF DESCRIPTION OF THE FIGURES

For a better understanding of the present invention, as well as other aspects and further features thereof, reference is made to the following description which is to be used in conjunction with the accompanying drawings, where:

FIG. 1 is a side cross sectional view of an embodiment of the absorbent composite material having absorbent core in the channels;

FIG. 2 is a cross sectional view of an embodiment of the absorbent composite material with an additional absorbent layer and backing layer;

FIG. 3 is a top perspective view of an absorbent article having an absorbent composite material in the upper layer; and

FIG. 4 is a perspective view of an absorbent article having an absorbent composite material in the upper layer with absorbent core in selected channels.

### DETAILED DESCRIPTION OF THE INVENTION

Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or element(s) as appropriate.

If the specification states a component or feature “may,” “can,” “could,” “preferably,” “possibly,” “optionally,” “for example,” “often,” or “might” or other such language be included or have a characteristic, that particular component or feature is not required to be included or to have the characteristic. Such component or feature may be optionally included in some embodiments, or it may be excluded.

The terms “fluid” and “body fluid” as used herein refer to fluids discharged from the body. Fluids may include but are not limited to urine, fluid fecal matter, menstrual fluid including blood and mucous, blood, wound secretions, breast milk and vaginal secretions.

The term “absorbent” is used herein in its normal meaning to refer to the property of a material to pick up and/or retain fluid.

The term “absorbent article” as used herein refers to a washable device or garment to be worn or used which is capable of absorbing body fluids. More specifically, the absorbent article is a device or garment that is placed in proximity to the body of the wearer or user to absorb and/or contain fluids discharged from the body. Absorbent articles may include diapers for infants and young children, incontinence garments ranging in size and shape from small pads to full diapers for children and adults, menstrual pads, training pants, adult feminine hygiene products, bandages, breast pads, care mats and wound dressing products. Other examples of absorbent articles include mattress pads, chair pads, furniture pads, cushions, and similar protective surfacing products. Absorbent articles may further include cleaning articles and fabrics such as rags and sponges, food industry articles, and the like.

The term “absorbent fibre” refers to a fibre that is capable of absorbing water or fluid. A variety of absorbent fibres are known, non-limiting examples of which include natural fibres such hemp, bamboo, cotton, silk, linen, flax and wool, and synthetic fibres such as polymers, polyester, rayon, cellulose, and all combinations thereof. A variety of washable absorbent fabrics can be made from absorbent fibres. Non-limiting examples of absorbent fabrics include flannel, shirting, wovens, non-wovens, terrycloth, fleece, felt, velour and velvet. Absorbent fibre can also be made into cording that is, for example, single ply, multiple ply, twisted, braided, spun or woven, or made into strips by cutting from a sheet of absorbent fibre material.

The term “washable” is used in its ordinary sense to mean a material or article that can be washed and re-used. Standard hand washing or machine washing or laundering may be used, optionally with air drying, which may include a clothes or fabric dryer or sun or air drying or a combination thereof.

The terms “bond” and “bonding” as used herein refer to any method by which two layers of fabric can be attached together in a secure manner. Bonding as used herein may include but is not limited to stitching, annealing, heat sealing, laminating and adhesive bonding.

The term “central region” as used herein refers to the region of an absorbent article which is expected to receive the most fluid or wetting. For example, in a diaper, menstrual pad or incontinence pad, the central region of the absorbent article is the area positioned between the legs of user or near the crotch area when the garment is worn.

Described is a washable absorbent composite material that can be used in the manufacture of absorbent articles to absorb body fluids. The presently described absorbent composite material is useful for making wearable absorbent articles such as menstrual pads, incontinence pads, feminine hygiene pads, infant diapers, child diapers, training pants, adult diapers, wound dressings, bandages, lactation (breast) pads and incontinence undergarments such as underwear. Other non-limiting examples of applications of the presently described absorbent composite material are mattress toppers or pads, chair cushions or pads, or in any other cover application where protection from fluid or wetting is advantageous.

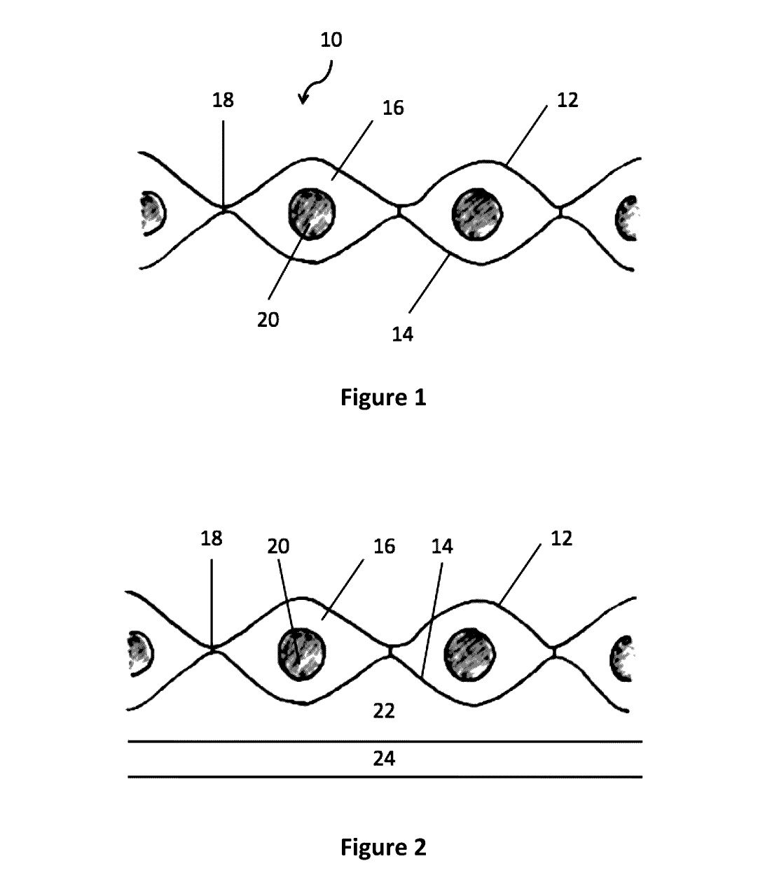

With reference to FIG. 1, an example absorbent composite material 10 comprises a first layer 12 of fluid permeable fabric and a second layer 14 of fluid permeable fabric bonded together with longitudinal channel binding 18 to form channels 16. Bonding of the first layer 12 and second layer 14 provides structure to the channels 16 while maintaining fluid transport capabilities between the first and second layers of fabric. Preferably the first layer, the second layer, or both comprise absorbent fabric.

A first or top layer 12 of the absorbent composite material is in contact with the body or skin of the user and should therefore provide a soft and comfortable surface. The first layer 12 is made from a fluid permeable and/or hydrophilic material capable of allowing fluid to pass through to the lower layers, absorb fluid, or both. Suitable fabrics for the first layer include but are not limited to woven and non-woven fabrics, knit fabrics and mesh materials. Optionally, the fabric of the first layer 12 can be subjected to a treatment or coating to add resilience, stain-resistance, antimicrobial or antiviral properties and/or improve fabric performance. In some embodiments, treatment of the first layer with a hydrophobic coating, such as, for example, teflon™, can promote permeability of fluids and reduce staining. As such, the first layer 12 has an affinity for and/or penetrability for fluids and, thus, readily absorbs moisture and/or allows moisture to easily pass through. The first layer can thus comprise a material that is water-permeable but less absorbent than the other layers. In this way, the first layer may encourage a flow of fluid from the outer surface of the material to the inner layer(s), inner core or both, trapping fluid inside the material while limiting the accumulation of fluid in the first layer and thus reducing surface wetness. In one non-limiting example the first layer comprises a polyester non-woven felt, such as polyethylene terephthalate felt, that enables passage of fluid between layers. The absorbent composite material can further comprise a mesh layer on top of the first layer which is in contact with the skin of the wearer. The mesh material of the mesh layer can serve as an additional barrier between the skin of the wearer and the first layer increasing air circulation between the skin of the wearer and the absorbent composite material. Preferably, the mesh comprises a non-absorbent material that enables fluids to pass through. The mesh layer may therefore decrease the feeling of wetness or dampness to the wearer while still enabling passage of fluids to the absorbent layers. Non-limiting materials from which the mesh can be made are polyester, rayon, and other polymers that have limited or no fluid absorbency. Some non-limiting examples of mesh material that can be used include athletic mesh and lace.

A second layer 14 disposed beneath or below the first layer 12 is made from a fluid permeable material that functions as a distribution or soaking layer for absorption, containment and distribution of fluid. The fabric of the first and second layers 12, 14 of the absorbent composite material can be the same or different, and can be a synthetic or natural fabric, either structured or unstructured. Preferably, the second layer comprises a woven, non-woven or knitted fabric made from absorbent fibres.

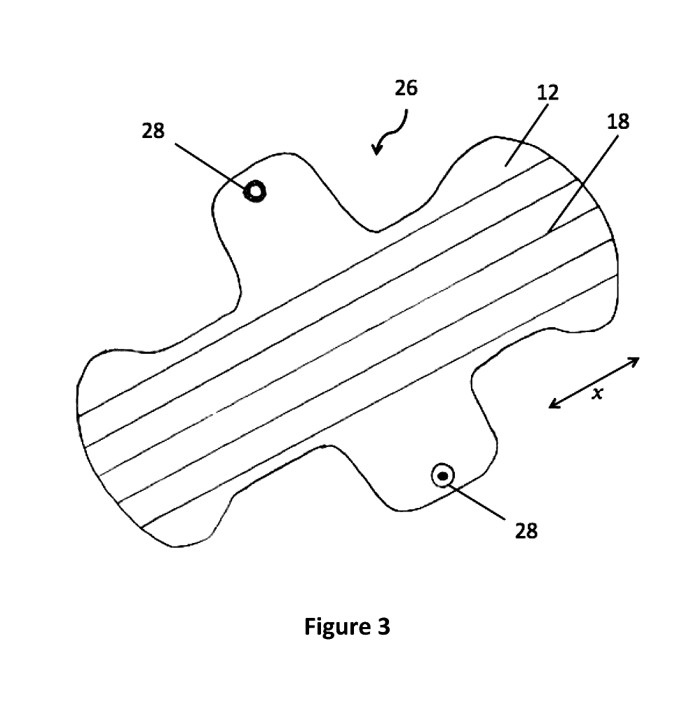

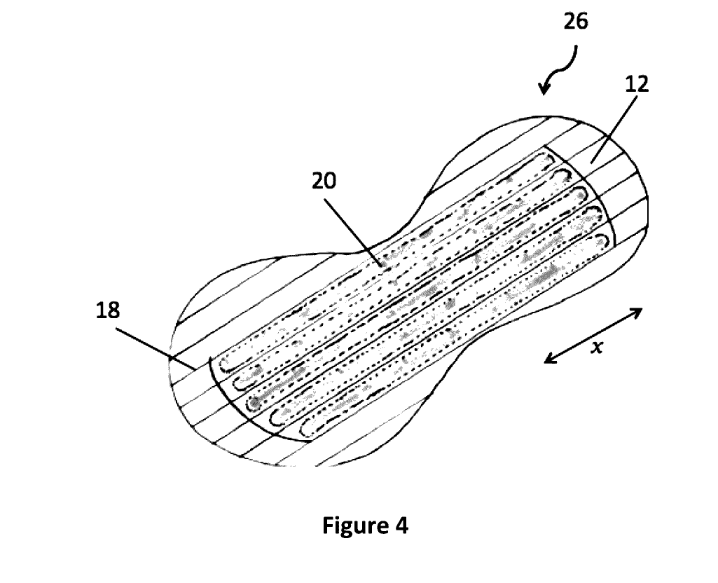

The first layer 12 and second layer 14 are bonded together with channel binding 18 to form longitudinal, substantially parallel channels 16, as shown in FIG. 3. Without wishing to be bound by theory, it is thought that the channels 16 in the absorbent composite material 10 provide stability to the absorbent composite material and assist in directing fluid flow through the material, from the surface layers to the interior layers and/or absorbent core. Channeling may also direct exudates and fluid along the longitudinal direction of the longitudinal channel binding 18, away from the fabric edges and into the material core. In particular, the channeled surface of the absorbent composite material directs moisture and fluid in a direction parallel to the channel binding. The channel binding 18 may also assist in directing fluid through the first layer 12 of the absorbent composite material and into lower fluid-absorbent layers by providing a point of contact between the first layer 12 and the lower layers of the absorbent composite material. In one example, where the channel binding is stitching, it has been found that the channel binding 18 directs fluid through the stitching and into lower absorbent layers. In an absorbent article as shown in FIGS. 3 and 4, positioning the channel binding along the longitudinal axis x of the article, so that the channel binding 18 extends from the front to the back of the article, directs fluid exudates toward the central region of the article and away from the article edges. Although the channel binding in the articles of FIGS. 3 and 4 is shown as parallel lines, it is possible for the channel binding to be in waves, curves, or other similar configuration, extending from the front to the back of the article.

The channel binding can be limited only to the central region of an article where the most wetting is expected as exemplified in FIG. 3, or can be throughout the article as exemplified in FIG. 4. When the absorbent composite material is used in an absorbent article, the number of channels is preferably at least two. In other configurations, absorbent articles can have three or more, four or more, five or more, or more than five channels.

The first layer 12 and bottom layer 14 can comprise one or more additional layers of absorbent or fluid permeable fabric for support and/or additional absorbency. Additional layers can be made from the same or different materials as the first layer and/or second layer, and/or can be comprised of thicker or more absorbent materials such as felt or fleece made from absorbent fibres. It is understood that if the absorbent composite material 10 comprises additional layers of fabric between the first layer and second layer, these can be incorporated into the composite material and secured to the other layers through the channel binding to provide a single absorbent composite material structure.

The channels 16 in the absorbent composite material formed by the channel binding 18 further provide linear structures that can assist with the absorption of fluid. The width of the channels can be constant across the fabric, and/or can vary depending on the requirements of the article for which the fabric is being used. For use in absorbent garments, the width of the channels is preferably between 0.5 and 10 cm, more preferably between 1.0 cm and 5.0 cm, and more preferably between 1.5 cm and 3.0 cm. The width of the channels can also vary within the fabric itself. For example, the channel width can alternate between wide and narrow widths, to accommodate the function of the article. In one example, the central region of a diaper or incontinence garment can have narrower channels to provide additional absorbency through the channel binding, and wider channels at the periphery. Alternatively, in another embodiment when the channels support an absorbent core, wider channels in the central region and narrower channels at the periphery can provide additional absorbency in the central region of the absorbent article.

As shown in FIG. 2, the channels 18 can further support an absorbent core 20 therein, which can provide additional absorbency in the absorbent composite material 10. Accordingly, one or more channels 16 in the composite absorbent material can be partially or completely filled with an absorbent core 20 to provide additional absorbency to the composite material. The absorbent core 20 can facilitate the absorption of fluid away from the first layer 12 and, more particularly, toward the second layer 14 of the absorbent composite material 10. The absorbent core can also facilitate containment of the absorbed fluid. More specifically, the absorbed fluid can be contained within the absorbent core and directed away from the first layer 12 and the skin of the user.

The absorbent core 20 can comprise a material or combination of materials capable of absorbing and/or containing fluid received onto the first layer 12 of the absorbent composite material. The absorbent core 20 in contact with the first layer 12 may also enable wicking of fluid from the first layer 12 into the absorbent core, reducing the moisture of the top layer of an absorbent article in contact with the user's skin. Preferably, the absorbent composite material comprises a breathable material. The absorbent core can thus draw moisture away from the first layer 12 and the user's body and absorb and/or disperse the moisture or fluid. In particular, the absorbed moisture can be dispersed into the absorbent core 20 and toward the bottom layer 14 of the absorbent composite material and contained therein, thereby enhancing the comfort and dryness of the user.

The absorbent core 20 can comprise any material that is capable of absorbing fluid. The absorbent core can allow fluid entering through the first layer to be drawn inwardly and retained in the absorbent composite material. In some non-limiting examples, the absorbent core may be a comprised of solid piece of absorbent material, such as, for example, a solid core of porous material such as sponge, wool, absorbent foam, roll or filament(s) of absorbent fabric, or a combination thereof. In another example, the absorbent core can comprise an absorbent particulate received in the channel, or absorbent particulate in a fluid permeable sleeve or cover which can be received in the channel. The absorbent core may also comprise an absorbent particulate material, a mixture of two or more absorbent particulate materials, or an absorbent particulate material in combination with an absorbent fibre. The absorbent core 20 of each channel in the article may be the same or different, depending on location of the channel in the article and the absorbency requirements of the article. In yet another example, the absorbent core can comprise a single sheet of fabric having substantially longitudinal strands of thickened material running there through around which the channel binding can be attached, with the first layer and second layer sandwiched on either side.

As the absorbent composite material and article made therefrom are designed to be washable, the absorbent core 20 must be dryable under ambient or clothes dryer conditions to regenerate the absorbent capacity of the core material and layer fabrics. For some materials capable of being used in the absorbent core, high temperature drying in a clothes dryer can assist in drying and/or desiccating the absorbent composite material comprising the absorbent core for re-use.

In one embodiment, the absorbent core may comprise silica gel beads. Silica is a naturally occurring mineral that can be processed in granular form as beads. Silica gel beads are a porous, nontoxic, food safe, non-friable, water-insoluble white solid that come in a variety of sizes. As a desiccant or absorbent, silica gel has pores which have an affinity for water molecules, making them capable of releasably retaining fluid. Once saturated with water, silica gel beads can be regenerated and the moisture therein removed for reuse by heating to about 120° C. (250° F.), such as in a clothes dryer or microwave. Silica gel beads come in a variety of diameters. For this application, the preferable diameter of silica gel beads is 1.0-5.0 mm, more preferably 2.0-4.0 mm.

In other preferred examples, the absorbent core 20 can be made from filaments of absorbent materials having a rope-like or long and thin shape such that the filaments can be received in the channels 16 formed by the channel binding 18. Non-limiting examples of materials that can be used include particulate or filaments comprising flexible, porous materials such as synthetic sponge, felted wool roving, cording or banding, polyester/rayon cording, or other absorbent materials. Filamentous material, i.e. having a long, thin shape, can be preferably used in some embodiments as accommodating the size and shape of the channels. With these materials, flexibility and compressibility can add to the comfort of the user while providing additional absorbency to the absorbent article 26. In making an absorbent composite material having an absorbent core 20 in the channels, the channels can either be pre-formed and filled with absorbent core afterward, or channel binding can be added around the absorbent core material during manufacture.

Channel and core configurations can be changed based on the function of the absorbent article. In one embodiment, the central region can have channels without absorbent core, or have only two channels, one on each side of the central region, containing absorbent core to prevent leakage of fluid. In another embodiment, multiple channels in the central region can contain an absorbent core, as shown in FIG. 4, to provide a highly absorbent article 26.

In some embodiments, as shown in FIG. 2, the absorbent composite material may be further bonded to a fluid repellent backing layer 24. The backing layer may prevent the fluid absorbed by the absorbent composite from soiling other articles that may contact the absorbent article, such as bed sheets, undergarments and other clothing. The backing layer 24 can be disposed under the absorbent layers and absorbent composite material 10 to prevent fluid leakage or wetting of adjacent fabrics. In an embodiment, a backing layer 24 is positioned adjacent to and beneath or below the absorbent layers. The backing layer 24 thereby functions as a barrier layer. In light absorbency applications such as where the absorbent article is designed to absorb smaller amounts of fluid, such as light menstrual flow or light incontinence, a fluid repelling material can be used. Non-limiting examples of suitable fluid repelling fabric include ripstop nylon, polyester fleece, urethane laminated fabrics, and other laminates, films and materials that provide a water repellent barrier layer. In such light use absorbent articles, the fluid repellent backing layer 24 can also be positioned under an area of the absorbent composite material receiving the most fluid, such as along the centreline of the central region of a mild incontinence pad. Alternatively, the fluid repellent backing layer can completely cover the surface area of the back of the article.

In other embodiments in high absorbency applications where an absorbent article is required to absorb more fluid, such as in a heavy incontinence pad, heavy menstrual flow pad or diaper, a backing layer 24 that is substantially impermeable to fluid is preferable. Suitable fluid impermeable backing layers include materials having urethane or vinyl films, laminates, and polymer sheeting materials such as, for example, vinyl, nitrile, neoprene, polyurethane, polystyrene, or other plastic or rubber. Natural products that are substantially fluid impermeable can also be used, such as oilcloth, fleece, and felted wool, optionally treated with fluid-repellent coatings. In high-absorbency applications, the backing layer 24 preferably comprises a temperature, abrasion, and/or degradation resistant film or layer such that the absorbent layers can be washed without significantly affecting the fluid resistance of the backing layer. In other preferable embodiments, the backing layer is a breathable and/or waterproof material. In such high-absorbency applications, the backing layer 24 can also have the same surface area as the absorbent composite material. As such, in high absorbency applications, the backing layer 24 may prevent absorbed moisture within the absorbent layers from passing therethrough.

In the manufacture of an absorbent article, an additional layer of absorbent material 22 comprising absorbent fibre can be positioned on top of the absorbent composite material, or between the absorbent composite material and the fluid repellent fabric to provide additional absorbency to the article.

According to certain embodiments, the absorbent article may be provided with a re-fastenable fastening system or fastening means 28 which can removably secure the absorbent article to a garment of the wearer, such as a pair of underwear or other undergarment, or secure the absorbent article to itself such that it can be removably retained on a garment of a user, such as a standard undergarment. One example fastening means, a snap system, is shown in FIG. 3. The re-fastenable fastening system may comprise one or more fastening means in the form of, for example, snaps, buttons, segments of velcro™ or other hook and eye system, or any other re-fastenable fastening means. In certain embodiments, the absorbent article can also comprise one or more tabs, straps or closures to secure the article to another garment, to itself such that it is positioned around a garment, or to a support system on the wearer such as, for example, a belt optionally with garters.

Washing of the absorbent composite material and articles made therefrom can be done after each use to wash fluids from the absorbent fabric and absorbent core and restore the absorbent article. Laundering in a washing machine is preferable, however washing can also be done by hand. In some embodiments, high temperature drying can assist in expelling fluids and wash water from the absorbent material and absorbent core, and can be done in an electric dryer, or in the sun. Depending on the washing methods available, different materials may be selected for the first and second layer and for the absorbent core to accommodate. For example, with access to high temperature electric dryers, high absorbency materials that require high temperatures to expel fluid can be used, for example desiccants that can be regenerated under moderately high temperature. When drying is more commonly done in the sun, materials that can be more easily dried are more advantageously used. Thorough drying between uses can prevent the growth of bacteria, fungi and other microorganisms, however additional antimicrobial fabric or antimicrobial treatment of fabric, the absorbent core, backing layer or other materials can also assist in the antimicrobial properties of the absorbent composite material.

Although the presently described absorbent composite material and articles made therefrom are intended to be washable, it is understood that the same or similar design can be applied to the manufacture of disposable materials and articles. For example, in a disposable article, the absorbent top layer and bottom layer of the composite material can be made of any suitable fluid-permeable material, including disposable materials. In addition, the channels may be filled with an absorbent, disposable polymer or polymeric material capable of absorbing body fluids, many of which are known to the skilled person.

All publications, patents and patent applications mentioned in this Specification are indicative of the level of skill of those skilled in the art to which this invention pertains and are herein incorporated by reference to the same extent as if each individual publication, patent, or patent application was specifically and individually indicated to be incorporated by reference.

The invention being thus described, it will be obvious that the same may be varied in many ways. Such variations are not to be regarded as a departure from the scope of the invention, and all such modifications as would be obvious to one skilled in the art are intended to be included within the scope of the following claims. The scope of the claims should not be limited by the examples provided in the specification but should be given the broadest interpretation consistent with the specification as a whole

1. A washable absorbent composite material comprising:
a first layer of fluid permeable fabric;
a second layer of fluid permeable fabric;
longitudinal channel binding attaching the first layer to the second layer forming channels therebetween; and
an absorbent core in at least one of the channels.

2. The composite material of claim 1, wherein the fluid permeable fabric of the first layer and the fluid permeable fabric second layer are absorbent.

3. The composite material of claim 1, wherein the absorbent core comprises at least one filament of absorbent fibre, synthetic sponge, felt, desiccant, absorbent particulate, or a combination thereof.

4. The composite material of claim 1, further comprising at least one additional layer of absorbent fabric between the first layer and the second layer.

5. A washable absorbent article comprising:
an absorbent composite material comprising a first layer of fluid permeable fabric and a second layer of absorbent fabric, the first layer and second layer attached with longitudinal channel binding forming channels therebetween; and
a fluid repellent backing layer bonded thereto.

6. The article of claim 5, wherein at least one of the channels comprises an absorbent core.

7. The article of claim 5, wherein the absorbent core comprises at least one filament of absorbent fibre, synthetic sponge, felt, desiccant, absorbent particulate, or a combination thereof.

8. The article of claim 5, further comprising a mesh layer on top of the first layer, wherein the channel binding extends through the mesh layer.

9. The article of claim 5, further comprising at least one additional layer of absorbent fabric between the first layer and the backing layer.

10. The article of claim 5, wherein the backing layer is substantially impermeable to fluid.

11. The article of claim 5, wherein the article is a menstrual pad, incontinence pad, feminine hygiene pad, infant diaper, child diaper, training pant, adult diaper, wound dressing, bandage, lactation pad, or incontinence undergarment.

12. A washable absorbent composite material comprising:
a first layer of fluid permeable fabric;
a second layer of fluid permeable fabric;
longitudinal channel binding attaching the first layer to the second layer forming channels therebetween; and
a fluid repellent backing layer bonded to the second layer.

13. The composite material of claim 12, further comprising a mesh layer on top of the first layer, wherein the channel binding extends through the mesh layer.

14. The composite material of claim 12, further comprising an absorbent core in at least one of the channels.

15. The composite material of claim 14, wherein the absorbent core comprises at least one filament of absorbent fibre, synthetic sponge, felt, desiccant, absorbent particulate, or a combination thereof.
